(19) United States
(12) Patent Application Publication
(10) Pub. No.: US 2007/0275119 A1
(43) Pub. Date: Nov. 29, 2007

(54) CONFECTIONERY COMPOSITIONS CONTAINING REACTABLE INGREDIENTS

(75) Inventor: Jamileh Lakkis, Morris Plains, NJ (US)

Correspondence Address: HOFFMANN & BARON, LLP, 6900 JERICHO TURNPIKE, SYOSSET, NY, 11791, US

(73) Assignee: CADBURY ADAMS USA LLC, Parsippany, NJ (US)

(21) Appl. No.: 11/754,514

(22) Filed: May 29, 2007

Related U.S. Application Data
(60) Provisional application No. 60/808,653, filed on May 26, 2006.

(51) Int. Cl. A23G 4/00

(52) U.S. Cl. 426/3; 426/74

(57) ABSTRACT

The present invention relates to compositions and comestibles containing ingredients capable of reacting with each other when consumed. More specifically, in some embodiments, the confectionery composition includes a first region including a first confectionery base and a source of calcium ions and a second region including a second confectionery base and a source of phosphate ions. Desirably, the confectionery composition is non-homogenous. The present invention also provides methods of preparing such confectionery compositions and methods of remineralizing teeth by administering such confectionery compositions to an individual.

### CROSS-REFERENCE TO RELATED APPLICATIONS

This application claims the benefit of U.S. Provisional Application No. 60/808,653, filed May 26, 2006, the contents of which are incorporated herein by reference.

### FIELD

The present invention includes compositions and comestibles containing ingredients capable of reacting with each other when consumed. The present invention also provides for methods of preparing such comestibles and methods of remineralizing teeth by administering such comestibles.

### BACKGROUND

Confectionery compositions can be used to deliver actives. For example, chewing gums that provide breath freshening and tooth whitening actives are widely available as are chewy candies that provide vitamins and minerals.

The residence time in the mouth (typically five to thirty minutes) of confectionery compositions often makes them well suited to deliver actives that require dwell time in the mouth for effectiveness.

In some instances, the actives provide a benefit when they react with each other. For example, remineralization of early caries lesions by supplying calcium and phosphate ions that precipitate on the tooth's surface to repair demineralized dentin has been attempted by adding calcium phosphates to chewing gums and candies. These attempts have been frustrated by limited release of the actives from the chewing gum and objectionable grittiness in the candies.

In some embodiments, creating effervescence in the mouth by supplying an acidic material and an alkaline material has been attempted by placing sodium bicarbonate in a powdered center filling of a confection. These attempts have been frustrated by the reaction proceeding prior to consumption of the confection.

It would, therefore, be desirable to deliver actives in a confectionery composition without encountering product defects or premature reactions in such a way that the actives are capable of reacting with each other in the mouth.

### SUMMARY

In some embodiments, a confectionery composition includes a first region that includes a first confectionery base and a source of calcium ions and a second region that includes a second confectionery base and a source of phosphate ions. In some embodiments, the first region and the second region contact the oral cavity substantially simultaneously upon consumption such that the calcium ions and the phosphate ions react with each other. In some embodiments, the confectionery composition is non-homogenous.

In some embodiments, a confectionery composition includes a first region that includes a hard candy and a calcium salt and the second region that includes a hard candy and a phosphate salt. In some embodiments, the first region and the second region contact the oral cavity substantially simultaneously upon consumption such that the calcium ions and the phosphate ions react with each other. In some embodiments, the confectionery composition is non-homogenous.

In some embodiments, a confectionery composition contains two or more reactable actives that react with each other in the mouth when the confectionery composition is consumed. In some embodiments, the confectionery composition is non-homogenous and the reactable actives are located in different regions of the confectionery composition and in still other embodiments, those different regions contact the oral cavity substantially simultaneously. In some embodiments, at least one of the regions provides an efficacy signal to the consumer of the confectionery composition.

In addition to containing reactable actives, in some embodiments, the confectionery composition can contain flavors. In some embodiments, each of the regions can contain flavors. In still other embodiments, the regions can contain flavors from the same or different flavor families. Also, the flavor intensity of the flavors can be manipulated such that in some embodiments, the flavor intensity in one region is different to the same as the flavor intensity of another region.

In embodiments with different regions, the regions can contain confectionery bases. These confectionery bases can include, but are not limited to, hard candy, soft candy, edible film, chewing gum, chocolate, and combinations thereof. In some embodiments, the confectionery bases of the different regions are the same while in other embodiments, the confectionery bases can be different.

In some embodiments, a method of making a confectionery composition includes the steps of forming a first confectionery region including a first confectionery base and a source of calcium ions, forming a second confectionery region including a second confectionery base and a source of phosphate ions, and combining the first confectionery region and the second confectionery region to form a non-homogenous confectionery composition.

In some embodiments, a method of remineralizing teeth includes administering confectionery compositions including or providing calcium and/or phosphate ions into the oral cavity of an individual.

In some embodiments, a method of creating an effervescent sensation includes administering confectionery compositions including or providing a substance whose pH is above 7.0 and/or a substance whose pH is below 7.0 into the oral cavity of an individual.

In some embodiments, a method of whitening teeth includes administering confectionery compositions including or providing a peroxyl compound and/or a compound whose pH is above 7.0 into the oral cavity of an individual.

In some embodiments, a kit for tooth remineralization includes confectionery compositions including or providing calcium and/or phosphate ions and instructions for use of the confectionery compositions along with a container housing the confectionery compositions and the instructions.

In some embodiments, an edible product kit includes a plurality of separate edible pieces, said plurality of edible pieces are packaged so that at least a first piece containing a first confectionery base and a source of calcium ions and a second piece containing a second confectionery base and a source of phosphate ions are contacted substantially simultaneously in the oral cavity, wherein the calcium ions and the phosphate ions react with each other in the mouth when consumed.

### DETAILED DESCRIPTION

As will be described in detail herein, some embodiments relate to confectionery compositions containing reactable actives wherein the reactable actives are separated by incorporation into different regions of a confectionery composition such that they react when the confectionery composition is consumed and the actives are released from the confectionery composition. These regions may be separate and distinct regions of the confectionery composition. Desirably, in some embodiments, each respective region is free of the opposing reactable active. In some embodiments, the different regions of the confectionery composition each have surfaces that are exposed to the oral cavity. With surface areas exposed to the oral cavity, each region will dissolve and release the reactable active contained in that region. Once released from their respective regions, the reactable actives react with each other.

The confectionery compositions can be configured in a variety of ways. For example, in some embodiments, the confectionery composition is homogeneous while in other embodiments, it is non-homogeneous. As used herein, the term “homogeneous” is used to mean confectionery compositions wherein different regions are intimately mixed together. In homogenous compositions, the reactable actives are interspersed in a single confectionery mass, such as, for example, a single hard candy carrier system. As used herein, the term “non-homogeneous” is used to mean confectionery compositions wherein different regions are not intimately mixed together. In non-homogenous compositions, the reactable actives are interspersed in separate confectionery masses. For example, in some embodiments, two separate hard candy carrier systems may be used. The separate confectionery masses form separate and discrete regions of the confectionery composition. For instance, the separate confectionery masses may be positioned adjacent to one another or the separate confectionery masses may form inner and outer regions of the confectionery composition.

In some embodiments, the different regions containing the reactable actives contact the oral cavity substantially simultaneously. To achieve this substantially simultaneous contact, in some embodiments, the different regions both form exterior surfaces while in other embodiments, none of the different regions form exterior surfaces. In still other embodiments, only one of the different regions forms an exterior surface.

In addition to delivering reactable actives, the different regions can contain flavors. In some embodiments, the flavors in the different regions can be from the same flavor family while in other embodiments, the flavors can be from different flavor families. As used herein, the term “flavor family” refers to flavors grouped into categories such as fruit, spice, mint, sweet, and savory. Each flavor family has a number of individual, distinct flavors but collectively they share similar characteristics. Also, the intensity of the flavors in the different regions can be manipulated. In some embodiments, the flavors in the different regions can have the same intensity while in other embodiments, the intensities can be different.

In addition to containing flavors, the different regions can provide efficacy signals to those that consume the confectionery compositions. In some embodiments, the efficacy signal can include, but is not limited to, compounds that provide cooling, warming, tingling, effervescence, salivation, and combinations thereof. In some embodiments, the efficacy signals in the different regions can be the same while in other embodiments, the efficacy signals can be different.

In some embodiments, the reactable actives are calcium and phosphate ions that may react to remineralize teeth when the confection is consumed. In some embodiments, the reactable actives are ingredients with ply below 7.0 and ingredients with pH above 7.0 that may react to neutralize the ingredients with pH below 7.0. In some embodiments, the reactable actives are peroxyl compounds and ingredients with pH above 7.0 that may react to whiten teeth. In some embodiments, the reactable actives are ingredients with pH above 7.0 and ingredients with pH below 7.0 that may react to effervesce when consumed. In some embodiments, ingredients providing a sensation to the consumer of the confectionery composition are included to indicate that the reaction is proceeding. In some embodiments, the sensation is cooling, warming, tingling, or salivation.

As used herein, the transitional term “comprising,” (also “comprises,” etc.) which is synonymous with “including,” “containing,” or “characterized by,” is inclusive or open-ended and does not exclude additional, unrecited elements or method steps, regardless of its use in the preamble or the body of a claim.

As used herein, the terms “reacting with” and “reactable” are used to describe actual or potential chemical reactions that may produce one or more reaction products as a result of the chemical reaction. The type of chemical reaction and the reaction product(s) are determined by the chemistry of the participating compounds and the conditions under which the reaction may proceed. For example, calcium and phosphate ions are reactable when they combine to form calcium phosphate which may then remineralize a tooth surface. Similarly, carbamide peroxide and sodium bicarbonate are reactable when they combine to form hydrogen peroxide which may whiten teeth. As a further example, citric acid and sodium bicarbonate are reactable when they combine to form carbon dioxide gas which may provide an effervescent sensation.

As used herein, the term “release rate” is used to mean the amount of an ingredient or compound that exits a comestible composition upon consumption of the comestible composition and is introduced into the oral cavity over time. The ingredient or compound may or may not be solubilized in saliva.

As used herein, the term “active” is used to mean any ingredient or compound added to the comestible composition to provide a benefit to the individual consuming the comestible composition. Such benefits can include, but are not limited to, tastes, flavors, sensations, therapeutic benefits, well being benefits, nutritional benefits, pharmaceutical benefits, emotional benefits, oral care benefits, and breath freshening benefits. Some examples of oral care actives include tooth remineralizing agents such as calcium and phosphate ions and tooth whitening agents such as peroxides.

As used herein, the term “confectionery base” includes any ingredient or group of ingredients that represent the bulk of the confectionery composition and provide the confectionery composition with its structural integrity and to which other ingredients are added. Examples of confectionery bases can include sucrose and corn syrup blends. Other examples can include blends of polyols, blends of polyols with sucrose and/or corn syrup. Still other examples can incorporate fats and hydrocolloids as in bases that include thin boiling starch mixed with sucrose, corn syrup and hydrogenated vegetable oil or bases that include pectin, sucrose, and corn syrup.

As used herein, the term “hard candy” is synonymous with “hard boiled candy,” “high boiled candy,” “drops,” and “lozenges,” and includes all confectionery compositions wherein the saccharide component(s) are heated to temperatures high enough to remove most of their moisture or where saccharide component(s) are blended without cooking such that the finished product moisture content is about five percent or less.

As used herein, the term “soft candy” includes all confectionery compositions containing more than about five percent moisture.

As used herein, the term “source” includes all ingredients, compounds, compositions, and materials containing at least one reactable compound.

As used herein, the term “efficacy signal” includes any taste, flavor, sensation, aroma, sound, or texture attribute of the comestible that communicates to an individual consuming or considering consuming the comestible that the comestible can provide a benefit. Examples of efficacy signals include cooling compounds such as menthol that signal cough suppression and/or throat soothing, food acids that signal mouth moistening, and tingling compounds such as jambu that signal tooth cleaning.

As used herein, the term “sensation” includes any perception occurring incident to consumption of the comestible that is not attributable to the comestible's taste alone. As used herein, the term “taste” includes perceptions of sweet, sour, salty, bitter, and umami.

As used herein, the term “remineralization” includes any mechanism by which the surface of teeth are replenished with minerals. Examples of remineralization include the action of calcium and phosphate ions at or on the tooth surface to form calcium phosphate and calcium phosphate complexes which can become incorporated into the mineral structure of the tooth surface.

### Reactable Actives

In some embodiments, the reactable actives include calcium and phosphate ions. Sources for calcium ions can include, but are not limited to, calcium gluconate, calcium lactate gluconate, calcium-boro gluconate, calcium citrate, calcium ascorbate, calcium lactobionate, calcium bromolactobionate, calcium malate citrate, calcium orotate, calcium pyruvate, calcium lactate, calcium carbonate, tricalcium phosphate, tricalcium citrate, calcium fumarate, calcium L lactate pentahydrate, calcium chloride, calcium sulfate, calcium glutarate, calcium hydroxide, calcium oxide, and combinations thereof.

Sources for phosphate ions can include, but are not limited to, phosphoric acid, calcium phosphate (mono-, di-, and tri-basic), sodium phosphate (mono-, di-, and tri-basic), disodium diphosphale, tetrasodiurn diphosphate, pentapotassiurm triphosphate, pentasodium triphosphate, sodium polyphosphate (Graham's salt), sodium hexametaphosphate, sodium potassium polyphosphate (Tammann's salt). Kurrol's salt (KPO₃)n, sodium tripolyphosphate, disodium phosphate, magnesium phosphate (mono-, di-, and tri-basic), potassium phosphate mono-, di-, and tri-basic), bone phosphate, ammonium phosphate, dibasic, ammonium polyphosphate, calcium polyphosphate, calcium pyrophosphate, potassium polyphosphate, potassium pyrophosphate, and combinations thereof.

In some embodiments, the calcium ion and phosphate ion sources are selected with similar release rates so that the remineralization reaction between the ions will occur on the tooth surface.

In some embodiments, the reactable actives are ingredients with pH below 7.0 and ingredients with pH above 7.0. Ingredients with pH above 7.0 can be characterized as being alkaline while ingredients with pH below 7.0 can be characterized as acidic. Ingredients with pH above 7.0 can include, but are not limited to, tripotassium phosphate, dipotassium phosphate, sodium bicarbonate, calcium hydroxide, sodium hydroxide, calcium fluoride, calcium phosphate, calcium sulphate, potassium chloride, potassium phosphate, calcium carbonate, activated carbon, alum, aluminum hydroxide, aluminum potassium sulfate, aluminum sodium sulfate, ammonium carbonate, ammonium bicarbonate, ammonium chloride, ammonium hydroxide, ammonium citrate, ammonium gluconate, ammonium sulfate, ammonium sulfite, ammonium sulfide, ammonium phosphate, calcium gluconate, calcium glycerophosphate, calcium hexametaphosphate, calcium peroxide, calcium hypophosphite, calcium sulfate, chlorine dioxide, copper gluconate, copper sulfate, ferric chloride, ferric phosphate, ferric pyrophosphate, ferric oxide, ferric sulfate, ferric sodium pyrophosphate, ferrous carbonate, ferrous gluconate, ferrous sulfate, hydrogen peroxide, magnesium carbonate, magnesium chloride, magnesium gluconate, magnesium glycerophosphate, magnesium hydroxide, magnesium phosphate, magnesium sulfate, manganese glycerophosphate, manganese hypophosphite, manganese sulfate, potassium carbonate, potassium bicarbonate, potassium chloride, potassium gluconate, potassium hydroxide, potassium glycerophosphate, potassium tripolyphosphate, sodium carbonate, sodium chloride, sodium hexametaphosphate, sodium hydroxide, sodium fluoride, sodium hypophosphite, sodium metaphosphate, sodium phosphate, sodium pyrophosphate, sodium sulfate, sodium sulfide, sodium sulfite, sodium tripolyphosphate, stannous chloride, stannic chlorite, zinc carbonate, zinc chloride, zinc oxide, zinc gluconate, sodium chlorite, and combinations thereof.

Ingredients with pH below 7.0 can include, but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid, glucono-delta-lactone, and combinations thereof.

In some embodiments, the ingredients with pH below 7.0 and the ingredients with pH above 7.0 are selected with similar release rates so that the neutralization reaction will occur in the mouth.

In some embodiments, the reactable actives are peroxy compounds and ingredients with pH above 7.0. Suitable peroxy compounds can include, but are not limited to, any orally acceptable compound(s) that delivers perhydroxy (OOH⁻) ions, such as hydrogen peroxide, peroxides of alkali and alkaline earth metals, organic peroxy compounds, and peroxy acids and salts thereof. Peroxides of alkali and alkaline earth metals can include, but are not limited to, lithium peroxide, potassium peroxide, sodium peroxide, magnesium peroxide, calcium peroxide, and barium peroxide. Organic peroxy compounds can include, but are not limited to, carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, monoperoxyphthalate and the like. Peroxy acids and their salts can include, but are not limited to, organic peroxy acids such as alkyl peroxy acids and monoperoxyphthalate, as well as inorganic peroxy acid salts including persulfate, dipersulfate, percarbonate, perphosphate, perborate and persilicate salts of alkali and alkaline earth metals such as lithium, potassium, sodium, magnesium, calcium and barium. Another useful peroxy compound is sodium pyrophosphate peroxyhydrate. Suitable ingredients with pH above 7.0 are listed above.

In some embodiments, the peroxy compounds and the ingredients with pH above 7.0 are selected with similar release rates so that the whitening or teeth cleaning reaction will occur in the mouth.

In some embodiments, the reactable actives are ingredients with pH above 7.0 and ingredients with pH below 7.0 that react to effervesce when consumed. Suitable ingredients with pH above 7.0 can include, but are not limited to, any orally acceptable bicarbonate such as alkali metal bicarbonates such as sodium and potassium bicarbonates, ammonium bicarbonate and the like. As discussed above, ingredients with pH below 7.0 can include, but are not limited to acetic acid, adipic acid, ascorbic acid, butyric acid, citric acid, formic acid, fumaric acid, glyconic acid, lactic acid, phosphoric acid, malic acid, oxalic acid, succinic acid, tartaric acid, glucono-delta-lactone, and combinations thereof.

In some embodiments, the ingredients with pH above 7.0 and ingredients with pH below 7.0 that react to effervesce are selected with similar release rates so that the effervescence reaction will occur in the mouth.

In some embodiments, ingredients providing a sensation to the consumer of the confectionery composition are included to indicate that the reaction is proceeding. In some embodiments, the sensation is cooling, warming, effervescence, tingling, or salivation. A variety of well known cooling agents may be employed. For example, among the useful cooling agents are included menthol, xylitol, erythritol, menthane, menthone, ketals, menthone ketals, menthone glycerol ketals, substituted p-menthanes, acyclic carboxamides, mono menthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, 2-isopropanyl-5-methylcyclohexanol, hydroxycarboxylic acids with 2-6 carbon atoms, cyclohexanamides, menthyl acetate, isopulegol, menthyl lactate, menthyl salicylate, N,2,3-trimethyl-2-isopropyl butanamide (WS-23), N-ethyl-p-menthane-3-carboxamide (WS-3), menthyl succinate, 3,1-menthoxypropane 1,2-diol, p-menthane-3,8-diols, and glutarate esters, among others. These and other suitable cooling agents are further described in the following U.S. patents, all of which are incorporated in their entirety by reference hereto: U.S. Pat. Nos. 4,230,688; 4032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether (TK-1000) supplied by Takasago Perfumary Company Limited, Tokyo, Japan, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethyleather, gingerol, shogaol, paradol, zingerone, capsaicin, dihydrocapsaicin, nordihydrocapsaicin, homocapsaicin, homodilhydrocapsaicin, ethanol, isopropyl alcohol, iso-amylalcohol, benzyl alcohol, glycerine, and combinations thereof.

The sensation of warming or cooling effects may be prolonged with the use of a hydrophobic sweetener as described in U.S. Patent Application Publication 2003/0072842 A1 which is incorporated in its entirety herein by reference. For example, such hydrophobic sweeteners include those of the formulae I-XI referenced therein. Perillartine may also be added as described in U.S. Pat. No. 6,159,509 also incorporated in its entirety herein by reference.

In some embodiments, a tingling sensation can be provided. One such tingling sensation is provided by adding jambu, oleoresin, sanshool, piperine, or spilanthol to some examples. In some embodiments, alkyiamides extracted from materials such as jambu or sanshool can be included. In some embodiments, compounds providing a tingling sensation can also provide a salivation sensation. One such salivation sensation is provided by adding trans-pellitorin. Other saliva inducing ingredients can include, but are not limited to extracts of Heliopsis longipes root, extracts of Piper nigrum, alkyloxyalkane acid amides, alk-2-en-4-yne acid amides, food acids, and compounds providing a salty taste.

In some embodiments, a sensation is created due to effervescence. Such effervescence is created by combining a basic material with an acidic material. In some embodiments, a basic material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of combinations of sensates that include “tingling” type sensates can be found in U.S. Pat. No. 6,780,443, the entire contents of which are incorporated herein by reference for all purposes. Combinations can include blends of cooling compounds, warming compounds, and tingling compounds such as a blend of menthol, capsicum oleoresin, and Jambu oleoresin.

In some embodiments where the reactable ingredients are included in separate regions of the confectionery composition, the ingredients that provide a sensation are included in one region. In some embodiments, the ingredients that provide a sensation are included in both regions of the confectionery composition. In some embodiments, the ingredients that provide a sensation will provide the same sensation while in other embodiments, the sensation will be different. In some embodiments, the ingredients that provide a sensation have similar release rates as compared to the release rates of the ingredients that react so that the reaction proceeds at the same time the sensation is provided in the mouth.

### Confectionery Forms & Methods of Making

The confectionery composition may be a comestible selected from forms such as, but not limited to, hard candy, soft candy, gummy candy, jelly candy, cotton candy, pressed tablets, edible film, lozenges, and the like. “Confectionery composition” includes all non-chewing gum confectionery compositions.

For example, some embodiments are directed to a comestible in the form of a lozenge or candy, also commonly referred to as confectioneries. Such confectionery compositions may include bulk sweeteners such as sugars and sugarless bulk sweeteners, or the like, or mixtures thereof. Bulk sweeteners generally are present in amounts of about 0.05% to about 99% by weight of the composition.

In some embodiments, high-intensity sweeteners also may be included in the confectionery compositions. Without being limited to particular sweeteners, representative categories and examples include:

(a) water-soluble sweetening agents such as dihydrochalcones, monellin, steviosides, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;

(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazin-4-one-2,2-dioxide (Acesulfame-K), the free acid form of saccharin, and mixtures thereof;

(c) dipeptide based sweeteners, such as L-aspartic acid derived sweeteners, such as L-aspartyl-L-phenylalanine methyl ester (Aspartame), N-[N-(3,3-dimethylbutyl)-L-α-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspaityl-2,5-dihydro-L-phenylalanine; L-aspartyl-L-(1-cyclohexen)-alanine, and mixtures thereof;

(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1chloro-1′-deoxysucrose; 4-chloro-4-deoxy-alpha-D-galactopyranosyl-alpha-D-fructoftiranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose;1′,6′˜dichloro1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose-4,6-dichloro-4,6-dideoxy-alpha-D-galactopyranosyl-6-chloro-6-deoxy-beta-D-fractofiranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro4,6,1′,6′-tetradeoxygalacto-sucrose; and 4,6,1′,6′-tetradeoxy-sucrose, and mixtures thereof;

(e) protein based sweeteners such as thaumaoccous danielli (Thaumatin I and II) and talin;

(f) the sweetener monatin (2-hydroxy-2-(indol-3-ylmethyl)-4-aminoglutaric acid) and its derivatives; and

(g) the sweetener Lo han guo (sometimes also referred to as “Lo han kuo”).

The intense sweetening agents may be used in many distinct physical forms well-known in the art to provide an initial burst of sweetness and/or a prolonged sensation of sweetness. Without being limited thereto, such physical forms include free forms, such as spray dried, powdered, beaded forms, encapsulated forms, and mixtures thereof.

In general, an effective amount of intense sweetener may be utilized to provide the level of sweetness desired, and this amount may vary with the sweetener selected. The intense sweetener may be present in amounts from about 0.001% to about 3%, by weight of the comestible, depending upon the sweetener or combination of sweeteners used. The exact range of amounts for each type of sweetener may be selected by those skilled in the art.

A variety of traditional ingredients also may be included in the confectioneries in effective amounts such as coloring agents, antioxidants, preservatives, and the like. Coloring agents may be used in amounts effective to produce the desired color. The coloring agents may include pigments which may be incorporated in amounts up to about 6%, by weight of the composition. For example, titanium dioxide may be incorporated in amounts up to about 2%, and preferably less than about 1%, by weight of the composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1comprises a triphenylrmethane dye and is the monosodium salt of 4-[4-(N-ethyl-p-sulfoniumbenzylamino)diphenylmethylene]-[1-(N-ethyl-N-p-sulfoniumbenzyl)-delta-2,5-cyclohexadieneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

Lubricants also may be added in some embodiments to improve the smoothness of the comestible, such as, for example hard candy embodiments, Smoothness also is a characteristic that leads to an increased perception of hydration upon consumption. Suitable lubricants include, but are not limited to, fats, oils, aloe vera, pectin, and combinations thereof.

Similarly, in some embodiments of the present invention, the comestible may have smooth edges. In such embodiments, the comestible may have any shape, such as square, circular or diamond-shaped, however, the edges are rounded to provide a smooth comestible. Another manner of lending smoothness to the comestibles is to deposit the comestible composition into molds during the manufacturing process. Accordingly, in some embodiments, the comestible is deposited.

Other conventional additives known to one having ordinary skill in the art also may be used in the confectionery compositions.

In some embodiments, confectionery compositions can be produced by batch processes. Such confections may be prepared using conventional apparatus such as fire cookers, cooking extruders, and/or vacuum cookers. In some embodiments, the bulk sweetener (sugar or sugar free) and a solvent (e.g., water), are combined in a mixing vessel to form a slurry. The slurry is heated to about 70° C. to 120° C. to dissolve any sweetener crystals or particles and to form an aqueous solution. Once dissolved, heat and vacuum are applied to cook the batch and boil off water until a residual moisture of less than about 4% is achieved. The batch changes from a crystalline to an amorphous, or glassy, phase. The delivery system(s) can then be admixed in the batch by mechanical mixing operations, along with any other optional additives, such as coloring agents, flavorants, and the like. The batch is then cooled to about 50° C. to 10° C. to attain a semi-solid or plastic-like consistency.

The optimum mixing required to uniformly mix the delivery system(s), flavors, colorants and other additives during manufacturing of hard confectionery is determined by the time needed to obtain a uniform distribution of the materials. Normally, mixing times of from four to ten minutes have been found to be acceptable. Once the candy mass has been properly tempered, it may be cut into workable regions or formed into desired shapes having the correct weight and dimensions. A variety of forming techniques may be utilized depending upon the shape and size of the final product desired. Once the desired shapes are formed, cool air is applied to allow the comestibles to set uniformly, after which they are wrapped and packaged.

Alternatively, various continuous cooking processes utilizing thin film evaporators and injection ports for incorporation of ingredients including the delivery system(s) are known in the art and can be used as well.

The apparatus useful in accordance with the present invention comprise cooking and mixing apparatus well known in the confectionery manufacturing arts, and selection of specific apparatus will be apparent to one skilled in the art.

### Edible Compositions—Pressed Tablets

In some embodiments, confectionery compositions in the form of pressed tablets such as mints may can be made by combining finely sifted sugar or sugar substitute, flavoring agent (e.g. peppermint flavor), binding agent such as gum arabic, and an optional coloring agent. The flavoring agent and binding agent are combined and then gradually the sugar or sugar substitute is added along with a coloring agent if needed.

The product is then granulated by passing through a seive of desired mesh size (e.g. 12 mesh) and then dried typically at temperatures of from about 55° C. to 60° C. The resulting powder is fed into a tableting machine fitted with a large size punch and the resulting pellets are broken into granules and then pressed.

Edible Compositions—Thin Films

In some embodiments, edible films that dissolve in the oral cavity can be used. Such films are made from various hydrocolloids including pullulan, starches, alginates, and combinations thereof.

In some embodiments, film-forming agents can include, but are not limited to, pullulan, hydroxypropylmethyl cellulose, hydroxyethyl cellulose, hydroxypropyl cellulose, polyvinyl pyrrolidone, methyl cellulose carboxymethyl cellulose, polyvinyl alchohol, polyethylene glycol, polyacrylic acid, methylmethacrylate copolymer, carboxyvinyl polymer, amylase, high amylase starch, hydroxypropylated high amylase starch, chemically modified starch, dextrin, indigestible dextrin, chitin, chitosan, levan, elsinan, collagen, zein, gluten, soy protein isolate, whey protein isolate, casein, and mixtures thereof. In some embodiments, other film forming agents can include hydrocolloids such as natural seaweeds, natural seed gum, natural plant exudates, natural fiber extracts, biosynthetic gums, gelatins, biosynthetic process starch or cellulosie materials, alginates, sodium alginate, calcium alginate, carrageenans, guar gum, locust gum, tara gum, gum arabic, acacia gum, ghatti gum, agar gum, xanthan gum, pectin, tragacanth gum, and combinations thereof. It will be readily recognized by those skilled in the art that these materials can serve more than one function in the invention. For example, in addition to film forming properties, many of these materials also function as binders, thickeners, and stabilizers.

In some embodiments, the edible film is prepared from an aqueous dispersion or solution of film forming agents. Film forming agents can include materials with affinities for water such that they swell and increase viscosity when introduced into water. In some embodiments, edible films are formed by introducing the film forming agents into water with mixing to prevent clumping. In some embodiments, other ingredients such as the reactable actives are incorporated into the dispersion or solution either before or after the addition of the film forming agents. After a homogeneous mixture of film forming agents, reactable actives and other additives such as sweeteners, flavors, and plasticizers is achieved, the solution is heated to remove excess moisture and cast upon a substrate for drying. The resulting matrix includes a structure including long and short chain polymers some of which have linear conformations and some of which have branched conformations. Upon drying, the edible film is formed and can be cut and/or packaged.

### Edible Compositions—Soft Confectionery

In some embodiments, the reactable actives can be used in various soft confectionery formats. Soft confectionery formats can include nougat, caramel, taffy, gummies, and jellies.

In some embodiments, a nougat composition can include two principal components, a high boiled candy and a frappe. By way of example, egg albumen or substitute thereof is combined with water and whisked to form a light foam. Sugar and glucose are added to water and boiled typically at temperatures of from about 130° C. to 140° C. and the resulting boiled product is poured into a mixing machine and beaten until creamy. The beaten albumen and flavoring agent are combined with the creamy product and the combination is thereafter thoroughly mixed.

In some embodiments, a caramel composition can include sugar (or sugar substitute), corn syrup (or polyol syrup), partially hydrogenated fat, milk solids, water, butter, flavors, emulsifiers, and salt. To prepare the caramel, the sugar/sugar substitute, corn syrup/polyol syrup, and water can be mixed together and dissolved over heat. Then, the milk solids can be mixed into the mass to form a homogeneous mixture. Next, the minor ingredients can be mixed in with low heat. The heat can then be increased to boiling. Once sufficient water is removed and color/flavor developed, the mass can be cooled somewhat and temperature sensitive ingredients (including the reactable actives) can be mixed in prior to discharging and forming/shaping/wrapping the finished product.

In some embodiments, a taffy composition can include sugar (or sugar substitute), corn syrup (or polyol syrup), partially hydrogenated fat, water, flavors, emulsifiers, and salt. The process for preparing taffy can be similar to that for caramel and, optionally, the final taffy mass can be pulled to develop its desired texture.

In some embodiments, a gummi composition can include sugar (or sugar substitute), corn syrup (or polyol syrup), gelatin (or suitable hydrocolloid), flavor, color, and optionally acid. The gummi can be prepared by hydrating the gelatin or suitable hydrocolloid, heating the sugar/corn syrup (sugar substitute/polyol syrup) and combining the two components with heat. Once the combined mixture reaches its final temperature or suitable sugar solids level, minor components such as flavor, color, the reactable actives, etc. can be incorporated into the mixture and then poured into molds prior to cooling, wrapping, and finishing. Various surface treatments such as applications of wax or fat can be applied to decrease sticking.

In some embodiments, a jelly composition can include a starch-based jelly or a pectin-based jelly. As with gummis, jelly products can be produced by hydrating the hydrocolloid and combining the hydrated mixture with a cooked syrup component. The mixture can then be cooked to a final moisture content and minor components can be incorporated (including the reactable actives). As with gummis, jelly candies can be poured into molds such as starch molds. As with gummis, surface treatments such as fats or waxes can be applied. Additionally, jelly candies can have dry surface treatments such as applications of sanding sugar, acid, non-pareils, and the like.

### Edible Compositions—Spun Sugar/Sugar Substitute

In some embodiments, an edible composition can be made by subjecting a carbohydrate matrix to melt spinning. Melt spinning is a process well known in the art of non-woven fiber and fabric manufacture. In melt spinning, a solid, polymeric material is melted to form a viscous liquid. This viscous liquid is then forced through a die with multiple small holes (also known as a spinneret) to form multiple filaments. The filaments then solidify upon cooling into fibers that can, depending on the polymeric starting material, be stretched to add strength. The fibers can then be further processed to form fabrics such as nylon, saran, and polyester. When a carbohydrate matrix forms the feedstock for the melt spinning process, a cotton-candy like web results. In some embodiments, the cotton candy like web can have reactable actives incorporated into it. Additionally, the cotton candy like web can be formed into discrete dosage units that resemble typical dosage forms such as tablets.

Melt spinning can be accomplished by any means well known in the art. In some embodiments, a cotton candy machine such as the Econo-Floss Model 3017 manufactured by Gold Medal Products Co., Cincinnati, Ohio is used. Any other apparatus or physical process which provides similar forces and temperature gradients can also be used.

Following melt spinning, the web can be manipulated to form a sheet. Manipulation can include, but is not limited to pulling, twisting, or entangling. In some embodiments, the manipulated sheet can then be formed into discrete dosage units. As used herein, the term “discrete dosage unit” refers to any format, such as tablets, discs, or lozenges, of the melt-spun web or dosage delivery vehicle that provides an end-user with an intended benefit. Discrete dosage units can be produced using conventional forming equipment following manipulation of the web or dosage delivery vehicle into a sheet. In some embodiments, the discrete dosage unit can be formed by passing the manipulated sheet of web or dosage delivery vehicle through a shaping roller to form a dosage unit in a conventional confectionery shape.

### Edible Compositions—Chocolate Confectionery

Chocolate processing begins with sorting, cleaning, and then roasting the cocoa seeds or beans. The seeds are then cracked to produce seed bits known as nibs. The nibs then undergo a sizing processing called winnowing. After winnowing, the nibs are then milled to form a thick paste known as chocolate liquor. The chocolate liquor can then be pressed to separate out cocoa butter with the remaining material being sized into chocolate powder. Chocolate crumb is formed by mixing chocolate liquor with condensed milk, cocoa fat, and sugar. Chocolate crumb becomes finished chocolate by refining through rollers followed by conching and tempering. Chocolate candies can be formed by mixing other ingredients such as caramel or nuts and other inclusions and forming the mass into finished product shapes which are packaged for sale. Many configurations and combinations are known to those in the art. The reactable actives can be added at any suitable point in the process. In addition to chocolate that meets the standard of identity for labeling the product as chocolate, in some embodiments, cocoa or chocolate containing compositions such as compound coatings and the like can be produced and can have the reactable actives added to them.

Further details regarding the preparation of confectionery compositions can be found in Skuse's Complete Confectioner (13^{th} Edition) (1957) including pp. 41-71, 133-144, and 255-262; and Sugar Confectionery Manufacture (2^{nd} Edition) (1995), E. B. Jackson, Editor, pp. 129-168, 169-188, 189-216, 218-234, and 236-258 each of which is incorporated herein by reference.

### Edible Compositions—Chewing Gum

In some embodiments, the reactable actives may be added to different regions of a chewing gum composition. Compositions of chewing gum are well known in the art and described in depth in, for instance, U.S. Pat. Nos. 6.685,916, 6,627,233, 6,685,916 and 6,696,044, the disclosures of which are incorporated by reference and some of which is summarized herein.

Chewing gum compositions typically include one or more of gum bases, flavoring agent and bulk sweeteners. Chewing gum also includes bubble gums.

The chewing gum compositions may be coated or uncoated and may be in the form of slabs, sticks, pellets, balls, compressed tablets, and the like. Compressed tablets are formed from compressible mixtures.

Center filled gum is another common chewing gum form. The gum portion has a similar composition and mode of manufacture to that described for chewing gum. However, the center fill is typically an aqueous solution or gel, which can be injected into the center of the gum during processing. The center fill gum may also be optionally coated and may be prepared in various forms such as in the form of a lollipop, pellet or slab.

The chewing gum composition includes gum base and most of the other typical chewing gum composition components such as sweeteners, softeners, flavoring agents and the like.

In some embodiments, the gum base generally includes elastomers, elastomer plasticizers, waxes, fats, oils, emulsifiers, fillers and texturizers. Elastomers constitute from about 5% to 95% by weight of the base, specifically 10% to 70% by weight and more specifically 15% to 45% by weight. Examples of elastomers include synthetic elastomers such as polyisobutylene, polybutylene, isobutylene-isoprene co-polymers, styrene-butadiene co-polymers, polyvinylacetate and the like. Elastomers may also include natural elastomers such as natural rubber as well as natural gums such as jelutong, lechi caspi, perillo, massaranduba balata, chicle, gutta hang kang or mixtures thereof. Other elastomers are known to those of ordinary skill in the art.

Elastomer plasticizers modify the firmness of the finished gum when used in the gum base. Elastomer plasticizers are typically present in an amount of up to about 75% by weight of the gum base, specifically from about 5% to 45% by weight and more specifically from about 10% to 30% by weight. Examples of elastomer plasticizers include natural rosin esters such as glycerol ester of partially hydrogenated rosin, glycerol ester of tall oil rosin, pentaerythnritol esters of partially hydrogenated rosin, methyl and partially hydrogenated methyl esters of rosin, and the like. Synthetic elastomer plasticizers such as terpene resins may also be employed in gum base composition.

Waxes include synthetic and naturally occurring waxes such as polyethylene, bees wax, carnauba and the like. Petroleum waxes such paraffin may also be used. The waxes may be present in the amount of up to about 30% by weight of the gum base. Waxes aid in the curing of the finished gum and help improve the release of flavor and may extend the shelf life of the product.

Fillers modify the texture of the gum base and aid processing. Examples of such fillers include magnesium and aluminum silicates, clay, alumina, talc, titanium oxide, cellulose polymners, and the like. Fillers are typically present in an amount of from 1% to 60% by weight.

Examples of softeners used in the gum base include hydrogenated and partially hydrogenated vegetable oils, cocoa butter, glycerol monostearate, glycerol triacetate, di- and triglycerides, fatty acids such as stearic acid, palmitic acid, oleic acid, linoleic acid, linolenic acid and the like.

The gum base constitutes between about 5% and 95% by weight of the chewing gum composition, more typically about 10% to 50% by weight, and most typically from about 25% to 35% by weight of the chewing gum composition.

Other ingredients used in chewing gum compositions include sweeteners, both natural and artificial and both sugar and sugarless. Sweeteners are typically present in the chewing gum compositions in amounts of from about 20% to 80% by weight, specifically from about 30% to 60% by weight. Sugarless sweeteners include, but are not limited sugar alcohols, such as sorbitol, mannitol, maltitol, xylitol, isomalt, erythritol, hydrogenated starch hydrolysates, and the like. High intensity sweeteners, such as sucralose, aspartame, neotame, salts of acesulfame, monatin, and the like are typically present in amounts of up to about 1.0% by weight.

Flavoring agents, which can vary over a wide range, may be selected in amounts from about 0.1% to 10.0% by weight, specifically from about 0.5% to 5.0% by weight. Flavoring agents for use in chewing gum compositions are well known and include citrus oils, peppermint oil, spearmint oil, oil of wintergreen, menthol, cinnamon, ginger and the like.

Softeners may be present to modify the texture of the chewing gum composition. As in typical gum compositions, softeners in the compositions are typically present in amounts of from about 0.5% to 10% by weight based on the total weight of the chewing gum composition.

Other materials, which may be present in the gum composition include antioxidants (e.g. butylated hydroxyanisole, butylated hydroxytoluene, beta-carotenes, tocopherols), colorants, flavoring agents and the like.

Coating techniques for applying a coating for a chewing gum composition such as pan and spray coating are well known. Preferred is coating with solutions adapted to build a hard candy layer. Both sugar and sugar alcohols may be used for this purpose together with high intensity sweeteners, colorants, flavoring agents, binders and other conventional additives.

The sweetener may be present in an amount of from about 30% to 80% by weight of the coating syrup. A binder such as magnesium stearate may be added to the coating syrup in an amount of from about 1% to 15% by weight of the coating syrup to enhance or promote adhesion. Optionally, minor amounts of conventional additives may also be present. The sweeteners suitable for use in the coating syrup include sugarless sweeteners such as the polyhydric alcohols, e.g., xylitol, sorbitol, mannitol, erythritol, isomalt, and mixtures, thereof; as well as maltitol, isomaltitol, hydrogenated starch hydrolysates, and hydrogenated glucose syrups. Mono, di- and polysaccharide may also be included. For example, sugars such as sucrose, fructose, glucose, galactose and maltose may also be employed as a sweetener. Other sweeteners suitable for use in the coating syrup include, but are not limited to free saccharin acid, water soluble salts of saccharin, cyclamate salts, palatinit dihydrochalcones, glycyrrhizin, L-asparyl-L-phenylalanine methyl ester, amino acid based sweeteners, talin, steviosides, dihydrochalcone compounds, acesulfame salts and mixtures thereof.

Other ingredients may be added in minor amounts to the coating syrup and include moisture absorbing compounds, anti-adherent compounds, dispersing agents and film forming agents. The moisture absorbing compounds suitable for use in the coating syrups include mannitol or dicalcium phosphate. Examples of useful anti-adherent compounds, which may also function as filler, include talc, magnesium trisilicate and calcium carbonate. These ingredients may be employed in amounts of about 0.5% to 5% by weight of the syrup. Examples of dispersing agents, which may be employed in the coating syrup, include titanium dioxide, talc or other anti-adherent compounds as set forth above.

The coating syrup can be heated and a portion thereof deposited on the chewing gum cores. Usually a single deposition of the coating syrup is not sufficient to provide the desired amount or thickness of coating and it usually will be desirable to apply second, third or more coats of the coating syrup in order to build up the weight and thickness of the coating to desired levels with layers allowed to dry in-between coats.

Further details regarding the preparation of chewing gum compositions can be found in Skuse's Complete Confectioner (13th Edition) (1957) including pp. 41-71, 133-144, and 255-262; and Sugar Confectionery Manufacture (2nd Edition) (1995), E. B. Jackson, Editor, pp. 258-286, the content of which is incorporated herein by reference.

The features and advantages of the present invention are more fully shown by the following examples which are provided for purposes of illustration, and are not to be construed as limiting the invention in any way.

### EXAMPLES

### Example 1

TABLE 1
Remineralizing composition

| | |
|---|---|
| Component | % by weight |

Calcium Region

| | |
|---|---|
| Sucrose | 40-60% |
| Glucose Syrup | 40-60% |
| Color | 0.005-0.05% |
| Acid | 0.05-0.5% |
| Flavor | 0.03-0.8% |
| Calcium Lactate | 0.25-1.5% |

Phosphate Region

| | |
|---|---|
| Sucrose | 40-60% |
| Glucose Syrup | 40-60% |
| Color | 0.005-0.05% |
| Acid | 0.05-0.5% |
| Flavor | 0.03-0.8% |
| Sodium Phosphate | 0.25-1.5% |

To form the confectionery product, the sucrose and glucose syrup for each region are dissolved in water, mixed, and heated to about 145° C. Next, solutions of calcium lactate and sodium phosphate are prepared by mixing with warm water. To the calcium region mass of cooked candy, the solution of calcium lactate is mixed. Likewise, to the phosphate region mass of cooked candy, the solution of sodium phosphate is mixed. The candy masses are then placed on cooling tables where color, acid, and flavor are mixed with kneading. The finished products are formed by combining the calcium region composition with an equal amount of the phosphate region, pressing the two regions together to form two layers and running the dual layered mass through a forming mechanism. Alternatively, the sugar and glucose syrup can be cooked and then placed on the cooling table for the addition of flavor, color, and acid. The batch can then be split for addition of calcium lactate to one region and addition of sodium phosphate to the other region. As before, a dual layer is then prepared and finished pieces are formed.

What is claimed is:

1. A confectionery composition comprising:
a first region comprising a first confectionery base and a source of calcium ions; and
a second region comprising a second confectionery base and a source of phosphate ions,
wherein said confectionery composition is non-homogenous.

2. The confectionery composition as in claim 1, wherein said first region and said second region contact the oral cavity substantially simultaneously upon consumption such that said calcium ions and said phosphate ions react with each other.

3. The confectionery composition as in claim 1, further comprising a coating.

4. The confectionery composition as in claim 1, wherein said first region and said second region both form exterior surfaces.

5. The confectionery composition as in claim 1, wherein neither said first region nor said second region form exterior surfaces.

6. The confectionery composition as in claim 1, wherein one of said first region or said second region forms an exterior surface.

7. The confectionery composition as in claim 1, wherein said first region further comprises a first flavor and said second region further comprises a second flavor.

8. The confectionery composition as in claim 7, wherein said first flavor is complementary to said second flavor.

9. The confectionery composition as in claim 7, wherein said first flavor is from the same flavor family as said second flavor.

10. The confectionery composition as in claim 7, wherein said first flavor is not from the same flavor family as said second flavor.

11. The confectionery composition as in claim 7, wherein the flavor intensity of said first flavor is greater than the flavor intensity of said second flavor.

12. The confectionery composition as in claim 7, wherein the flavor intensity of said first flavor is not greater than the flavor intensity of said second flavor.

13. The confectionery composition as in claim 2, wherein the weight percent of said first region is substantially equal to the weight percentage of said second region.

14. The confectionery composition as in claim 2, wherein the weight percent of said first region is not substantially equal to the weight percent of said second region.

15. The confectionery composition as in claim 1, wherein said first confectionery base is selected from the group consisting of hard candy, soft candy, edible film, chewing gum, and chocolate.

16. The confectionery composition as in claim 15, wherein said first confectionery base further comprises a sugar, a polyol, or combinations thereof.

17. The confectionery composition as in claim 1, wherein said second confectionery base is selected from the group consisting of hard candy, soft candy, edible film, chewing gum, and chocolate.

18. The confectionery composition as in claim 17, wherein said second confectionery base further comprises a sugar, a polyol, or combinations thereof.

19. The confectionery composition as in claim 1, wherein said first confectionery base is the same as said second confectionery base.

20. The confectionery composition as in claim 1, wherein said first confectionery base is not the same as said second confectionery base.

21. The confectionery composition as in claim 1, wherein at least one of said first region and said second region provides an efficacy signal to a consumer of said confectionery composition.

22. The confectionery composition as in claim 21, wherein said efficacy signal is selected from the group consisting of a cooling sensation, a warming sensation, a tingling sensation, an effervescence sensation, a salivation sensation, and combinations thereof.

23. The confectionery composition as in claim 21, wherein said first region provides a first efficacy signal to a consumer of said confectionery composition and said second region provides a second efficacy signal to said consumer.

24. The confectionery composition as in claim 23, wherein said first efficacy signal and said second efficacy signal are independently selected from the group consisting of a cooling sensation, a warming sensation, a tingling sensation, an effervescence sensation, a salivation sensation, and combinations thereof.

25. The confectionery composition as in claim 23, wherein said first efficacy signal is the same as said second efficacy signal.

26. The confectionery composition as in claim 23, wherein said first efficacy signal is not the same as said second efficacy signal.

27. A confectionery composition comprising:
a first region comprising a hard candy base and a calcium salt; and
a second region comprising a hard candy base and a phosphate salt;
wherein said first region and said second region contact the oral cavity substantially simultaneously upon consumption such that said calcium ions and said phosphate ions react with each other, and wherein said confectionery composition is non-homogenous.

28. A confectionery composition comprising:
a first region comprising a first reactable active; and
a second region comprising a second reactable active, wherein said first reactable active and said reactable second active react with each other in the mouth when said confectionery composition is consumed,
wherein at least one of said first region and said second region provides an efficacy signal to a consumer of said confectionery composition, and wherein said confectionery composition is non-homogenous.

29. The confectionery composition as in claim 28, wherein said first region and said second region contact the oral cavity substantially simultaneously when the confectionery composition is consumed.

30. The confectionery composition as in claim 28, wherein said first reactable active comprises a substance whose pH is above 7.0 and said second reactable active comprises a substance whose pH is below 7.0.

31. The confectionery composition as in claim 30, wherein said substance whose pH is above 7.0 comprises a bicarbonate salt.

32. The confectionery composition as in claim 28, wherein said first reactable active comprises a peroxyl compound and said second reactable active comprises a substance whose pH is above 7.0.

33. The confectionery composition as in claim 32, wherein said peroxyl compound is selected from the group consisting of hydrogen peroxide, carbamide peroxide, or combinations thereof.

34. The confectionery composition as in claim 28, wherein said efficacy signal is selected from the group consisting of a cooling sensation, a warming sensation, a tingling sensation, an effervescence sensation, a salivation sensation, and combinations thereof.

35. A method of making a confectionery composition comprising the steps of:
forming a first confectionery region comprising a first confectionery base and a source of calcium ions;
forming a second confectionery region comprising a second confectionery base and a source of phosphate ions; and
combining said first confectionery region and said second confectionery region to form a non-homogenous confectionery composition.

36. The method of making a confectionery composition as in claim 35, further comprising the step of applying a coating to said confectionery composition.

37. The method of making a confectionery composition as in claim 35, wherein said first confectionery region and said second confectionery region contact the oral cavity substantially simultaneously when said confectionery composition is consumed.

38. A method of remineralizing teeth comprising the step of administering the confectionery composition of claim 1 into the oral cavity of an individual.

39. A method of creating an effervescent sensation comprising the step of administering the confectionery composition of claim 30 into the oral cavity of an individual.

40. A method of whitening teeth comprising the step of administering the confectionery composition of claim 32 into the oral cavity of an individual.

41. A kit for tooth remineralization comprising:
the confectionery composition of claim 1;
instructions for use of said confectionery composition; and
a container housing said confectionery composition and said instructions for use.

42. An edible composition kit comprising a plurality of edible pieces, a package wherein said plurality of edible pieces are packaged so that at least a first piece and a second piece are capable of contacting the oral cavity substantially simultaneously, wherein said first piece comprises a first confectionery base and a source of calcium ions and said second piece comprises a second confectionery base and a source of phosphate ions, wherein said calcium ions and said phosphate ions react with each other in the mouth when consumed.
